(19) United States
(12) Patent Application Publication
(10) Pub. No.: US 2022/0062018 A1
(43) Pub. Date: Mar. 3, 2022

(54) Hip Brace Kit

(71) Applicant: Outcome-Based Technologies LLC, Cheyenne, WY (US)

(72) Inventor: Donald P. LoGuercio, Scottsdale, AZ (US)

(73) Assignee: Outcome-Based Technologies LLC, Cheyenne, WY (US)

(21) Appl. No.: 17/464,153

(22) Filed: Sep. 1, 2021

Related U.S. Application Data
(60) Provisional application No. 63/073,441, filed on Sep. 1, 2020.

(51) Int. Cl. A61F 5/01

(52) U.S. Cl. CPC A61F 5/0193; A61F 2005/0183; A61F 2005/0165; A61F 5/0102

(57) ABSTRACT

A hip brace kit includes a first assembly for retention against a limb, which has a first base and a first arm terminating at first rings. A second assembly includes a second base and a second arm terminating at a second ring. A third assembly includes a third base and a third arm terminating at a third ring. A pivot assembly includes an upper cup and a lower cup defining a pivot housing. In a first arrangement, the first and second assemblies are mounted in the pivot assembly to enable unrestricted pivotal movement with respect to each other and to disable adduction and abduction with respect to each other. In a second arrangement, the first and third assemblies are mounted in the pivot assembly to disable unrestricted pivotal movement with respect to each other and to enable adduction and abduction with respect to each other.

### CROSS-REFERENCE TO RELATED APPLICATIONS

This application claims the benefit of U.S. Provisional Application No. 63/073,441, filed Sep. 1, 2020, which is hereby incorporated by reference.

### FIELD

The present specification relates generally to medical devices, and more particularly to post-operative care and therapeutic medical devices.

### BACKGROUND

There are many types of post-operative orthotic devices from which doctors and patients may choose. Likewise, there are many types of pain management devices available to aid recovery after surgery. All devices have particular features and drawbacks. Improved devices are always needed, to enable or limit movement in certain directions, to control temperature at a surgical site, to apply or remove pressure on a body part, to rotate, abduct, or adduct a limb, to support or add weight, to stabilize joints, and for various other.

### SUMMARY

In an embodiment, a hip brace kit includes a first assembly for retention against a limb, which has a first base and a first arm terminating at first rings. A second assembly includes a second base and a second arm terminating at a second ring. A third assembly includes a third base and a third arm terminating at a third ring. A pivot assembly includes an upper cup and a lower cup defining a pivot housing. In a first arrangement, the first and second assemblies are mounted in the pivot assembly to enable unrestricted pivotal movement with respect to each other and to disable adduction and abduction with respect to each other. In a second arrangement, the first and third assemblies are mounted in the pivot assembly to disable unrestricted pivotal movement with respect to each other and to enable adduction and abduction with respect to each other.

In an embodiment, a hip brace kit includes a first assembly for bracing against a leg, which includes a base and an arm terminating at a pivot connection defined by concentric rings. A second assembly is for bracing against the leg and includes a base and an arm terminating at a pivot connection defined by a ring. A third assembly is for bracing against the leg and includes a base and an arm terminating at a pivot connection defined by a ring. The hip brace kit has a first arrangement in which the first and second assemblies are mounted in a pivot assembly to enable unrestricted pivotal movement and to disable adduction and abduction. The hip brace kit has a second arrangement in which the first and third assemblies are mounted in the pivot assembly to disable unrestricted pivotal movement and to enable adduction and abduction.

The above provides the reader with a very brief summary of some embodiments described below. Simplifications and omissions are made, and the summary is not intended to limit or define in any way the disclosure. Rather, this brief summary merely introduces the reader to some aspects of some embodiments in preparation for the detailed description that follows.

### BRIEF DESCRIPTION OF THE DRAWINGS

Referring to the drawings:

FIGS. 1A and 1B are top and bottom perspective views of a hip brace kit;

FIG. 2 is an exploded top perspective view of the hip brace kit;

FIGS. 3A and 3B are section views of the hip brace kit taken along the line 3-3 in FIG. 1A;

FIGS. 4A-4D illustrate steps of removing an upper assembly of the hip brace kit;

FIGS. 5A-5C illustrate steps of re-assembling the hip brace kit with new elements;

FIGS. 6A and 6B are top and bottom perspective views of the hip brace kit with the new elements;

FIGS. 7A and 7B are section views of the hip brace kit taken along the line 7-7 in FIG. 6A; and

FIG. 8 is an enlarged section view through the hip brace kit along the line 8-8 in FIG. 6A.

### DETAILED DESCRIPTION

Reference now is made to the drawings, in which the same reference characters are used throughout the different figures to designate the same elements. Briefly, the embodiments presented herein are preferred exemplary embodiments and are not intended to limit the scope, applicability, or configuration of all possible embodiments, but rather to provide an enabling description for all possible embodiments within the scope and spirit of the specification. Description of these preferred embodiments is generally made with the use of verbs such as “is” and “are” rather than “may,” “could,” “includes,” “comprises,” and the like, because the description is made with reference to the drawings presented. One having ordinary skill in the art will understand that changes may be made in the structure, arrangement, number, and function of elements and features without departing from the scope and spirit of the specification. Further, the description may omit certain information which is readily known to one having ordinary skill in the art to prevent crowding the description with detail which is not necessary for enablement. Indeed, the diction used herein is meant to be readable and informational rather than to delineate and limit the specification; therefore, the scope and spirit of the specification should not be limited by the following description and its language choices.

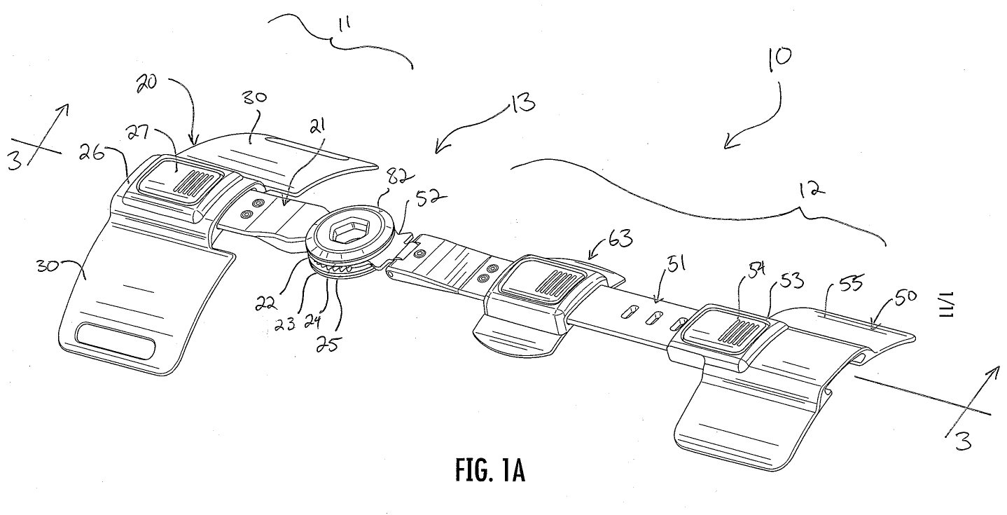

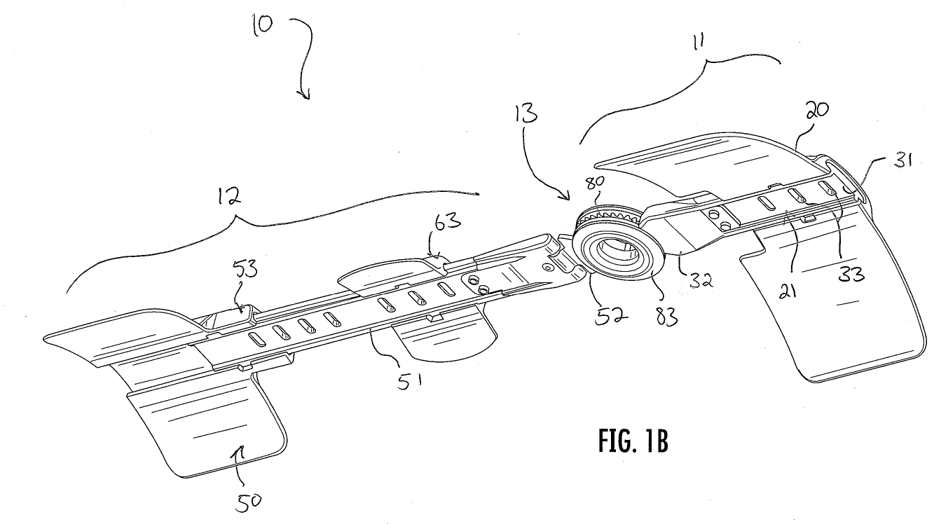

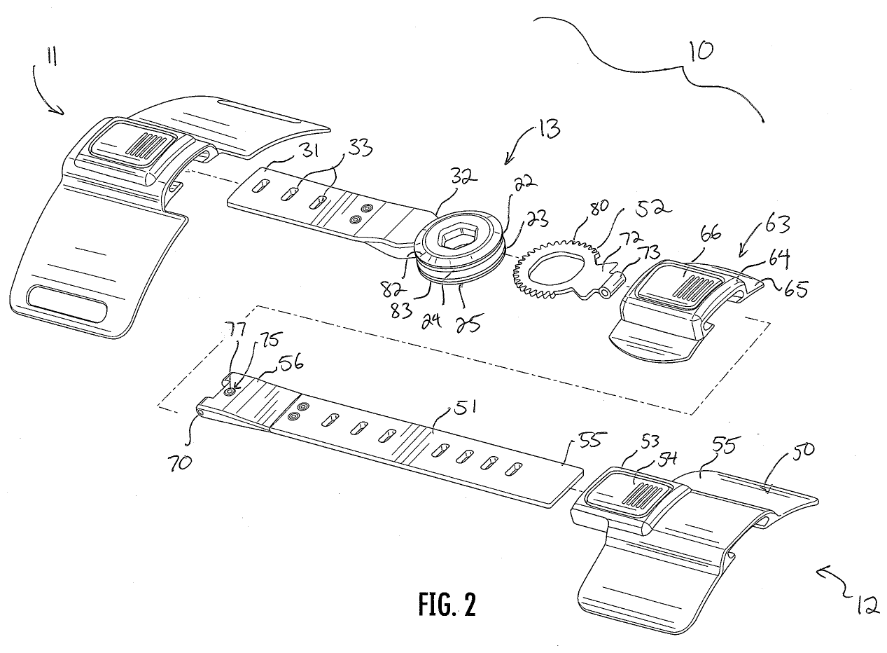

Reference now is made to the drawings, in which the same reference characters are used throughout the different figures to designate the same elements. FIGS. 1A and 1B are top and bottom perspective views of a hip brace kit 10 (hereinafter, the “kit 10”), and FIG. 2 is an exploded view of the kit 10. The kit 10 includes a first or upper assembly 11 and a second or lower assembly 12 mounted at a pivot assembly 13 for a movement through different ranges and planes of motion. The kit 10 is worn on the hip and leg to allow and restrict those different movement types.

The pivot assembly 13 enables a free, unrestricted range of motion of the upper and lower assemblies 11 and 12 in a single plane, but disables adduction and abduction of the upper and lower assemblies out of that plane. However, portions of the kit 10 are replaceable to achieve different functionality. When the kit 10 is modified, the upper and lower assemblies 11 and 12 can be adjustably limited in pivotal movement to disable unrestricted pivotal movement of the first and second assemblies 11 and 12 and thus restrict flexion and extension of the patient's hip beyond desired angles, and can be adjustably set to enable adduction or abduction of the first and second assemblies 11 and 12 and thus provide a desired adduction or abduction of the leg with respect to the hip. In this way, the kit 10 provides an orthotic with multiple configurable arrangements that can be modified to fit a patient's particular post-operative recovery needs.

The upper assembly 11 includes a base 20, an arm 21 extending downward from the base 20 and terminating at upper and lower rings 22 and 23 opposite the base 20 and carried within the pivot assembly 13, and spacers 24 and 25 also within that pivot assembly 13. The base 20 is a thin, wide, rigid base for application and retention against the lateral portion of a patient's hip. The base 20 has a central buckle 26 with a top button 27. The arm 21 slides into and engages with the buckle 26 to be releasably secured in the base 20. Opposed wings 30 extend laterally outward from the buckle 26 and are curved slightly to correspond to a patient's hip. Slots formed through the distal ends of the wings 30 receive a belt or other structure to secure the kit 10 about a patient's hip. In other embodiments, the underside of the base 20 carries a hook-and-loop engagement element for securing to a complemental hook-and-loop engagement element on a belt or soft pad wrapped around the patient.

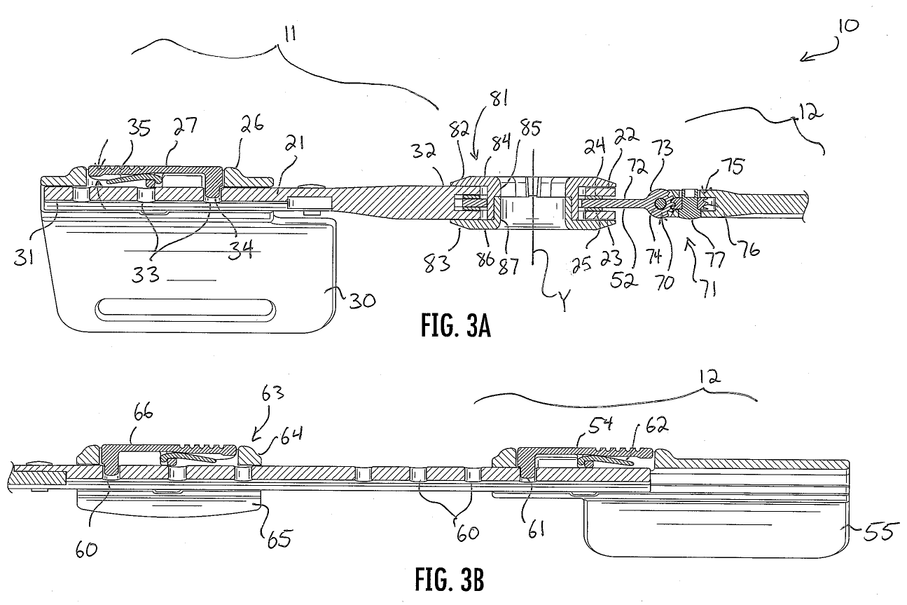

The arm 21 extends between the pivot assembly 13 and the base 20 to set the desired offset location of the pivot assembly 13 with respect to the base 20. The arm 21 is thin, elongate, and rigid. It includes an upper end 31 and an opposed lower end 32. As shown in the section view of FIG. 3A, several spaced-apart slots 33 are formed through the upper end 31 of the arm 21. The underside of the lower end of the button 27 has a tooth 34 which projects inwardly through one of the slots 33 to hold the arm 21 with respect to the buckle 26. The upper end of the button 27 can be depressed to pivot the button 27 and lift the tooth 34, which disengages the arm 21 from the buckle 26; the arm 21 can then be moved upward or downward, as desired, within the buckle 26 to change the effective length of the arm 21 and thereby move the pivot assembly 13 with respect to the base 20. A flat spring 35 is compressed between the upper end 31 of the arm 21 and the button 27; the flat spring 35 biases the button 27 into the engaged position so that the button 27 does not inadvertently come loose and disengage the arm 21.

The lower end 32 of the arm 21 transitions into and terminates at the upper and lower rings 22 and 23. Referring primarily to FIGS. 1A, 2, and 3A, the rings 22 and 23 are coaxial, coextensive, parallel to each other, and vertically spaced apart from each other defining a gap therebetween. The rings 22 and 23 project off the lower end 32 as two separated prongs of the arm 21. The two spacers 24 and 25 are held between the rings 22 and 23, and a portion of the lower assembly 12 is then disposed between the spaces 24 and 25, all cooperating within the pivot assembly 13 to enable or disable movement in different planes of motion.

The lower assembly 12 is best shown in FIGS. 1A, 1B, 2, and 3B. The lower assembly 12 includes a base 50, an arm 51 extending upward from the base 50 and terminating at a ring 52 opposite the base 50 and carried within the pivot assembly 13. The base 50 is a thin, wide, rigid base for application and retention against the outside or lateral portion of a patient's thigh. The base 50 has a central buckle 53 with a top button 54. The arm 51 slides into and engages with the buckle 53 to be releasably secured in the base 50. Opposed wings 55 extend laterally outward from the buckle 53 and are curved slightly to correspond to the patient's body. The wings 55 may be secured against a patient's thigh with an adjustable and/or elastic strap, or preferably, the underside of the base 50 carries a hook-and-loop engagement element for securing to a complemental hook-and-loop engagement element.

The arm 51 extends between the pivot assembly 13 and the base 50 to set the desired offset location of the base 50 with respect to the pivot assembly 13. The arm 51 is thin, elongate, and rigid. It includes an upper end 56 and an opposed lower end 57. As shown in the section view of FIG. 3, several spaced-apart slots 60 are formed through the arm 51. The underside of the upper end of the button 54 has a tooth 61 which projects inwardly through one of the slots 60 to hold the arm 51 with respect to the buckle 53. The lower end of the button 54 can be depressed to pivot the buckle 53 and lift the tooth 61, which disengages the arm 51 from the buckle 53; the arm 51 can then be moved into and out of the buckle 53, as desired, to change the effective length of the arm 51 and thereby move the base 50 with respect to the pivot assembly 13. A flat spring 62 is compressed between the arm 51 and the button 54; the flat spring 62 biases the button 54 into the engaged position so that the button 54 does not inadvertently come loose and disengage the arm 51.

A secondary base 63 is located between the upper and lower ends 56 and 57 of the arm 51. The secondary base 63 has a central buckle 64, laterally-extending wings 65, and a depressible button 66 mounted in the buckle 64. The base 63 is mounted over the arm 51 for sliding movement, and a tooth in the button 66 engages with the slots 60 in the arm 51. The base 63 acts as a thigh bolster, providing additional leverage during abduction. Preferably, the underside of the base 63 carries a hook-and-loop engagement element for securing to a complemental hook-and-loop engagement element.

Referring primarily to FIGS. 2-3B, the upper end 56 terminates in a cylindrical slot 70 transverse to the length of the arm 51. An adjustment assembly 71 is carried at the slot 70. The adjustment assembly 71 includes the ring 52, a short arm 72 extending downward from the ring 52, and a lateral knuckle 73. The knuckle 73 is mounted on a pin 74 carried in the cylindrical slot 70, and the knuckle 73 has teeth directed toward the upper end 56 of the arm.

Behind the cylindrical slot 70 is a socket 75, and a small worm screw 76 is carried within the socket 75. The worm screw 76 has a perimeter formed with outwardly-directed threads. Those threads are complemental to the teeth on the knuckle 73. The worm screw 76 is mounted on an adjustment knob 77, which extends out of a hole in the upper end 56 of the arm 51 where it can be accessed. Rotating the knob 77 in one direction or another imparts rotational movement to the worm screw 76, whose threads engage with the teeth on the knuckle 73 and thereby pitch the knuckle 73 up or down. Because the ring 52 is rigidly connected to the knuckle 73 by the arm 72, pitching movement of the knuckle imparts pitching movement to the ring 52. The ring 52 is applied to the pivot assembly 13, and so pitching movement of the ring 52 causes the lower assembly 12 to move into and out of the plane of the pivot assembly 13. Thus, the adjustment assembly 71 can be adjusted to set a desired angle of the lower assembly 12 with respect to the pivot assembly 13, thereby defining a desired degree of hip abduction or adduction.

The ring 52 cooperates with the rings 22 and 23, and the spacers 24 and 25 to define part of the pivot assembly 13 of the kit 10. The ring 52 itself is a pinion ring: an annular ring with an inner diameter corresponding to the inner diameters of the rings 22 and 23, and with an outer circumference formed with outwardly-directed teeth 80.

The rings 22 and 23 pivot or move in rotational movement, within a pivot housing 81 of the pivot assembly 13. Referring primarily to FIG. 3A, the pivot housing 81 is formed by an upper cup 82 and a lower cup 83. The upper cup 82 includes an annular flat top 84 and a central neck 85 extending downward in the hollow space in the middle of the upper and lower rings 22 and 23. The central neck 85 is formed with internally-directed threads to engage with the lower cup 83.

The lower cup 83 has an annular flat bottom 86 and a central neck 87 extending upward into the hollow space in the middle of the rings 22 and 23. The neck 87 of the lower cup 83 has an outer diameter corresponding to the inner diameter of the neck 85 of the upper cup 82, and the neck 87 of the lower cup 83 includes outwardly-directed threads complemental to those on the neck 85 of the upper cup 82. The upper and lower cups 82 and 83 thus threadably engage to each other to form the pivot housing 81. When so engaged, the pivot housing 81 defines an annular channel or hold 90 around the pivot housing 81 with a central axis Y. The upper and lower rings 22 and 23 are received in the hold 90, as is the pinion ring 52, with the spacers 24 and 25 disposed therebetween as bearing surfaces.

As shown in FIG. 3A, the top 84 of the upper cup 82 laps over the upper ring 22 and defines an upper bound of the annular hold 90. Below the top 84 is the upper ring 22 of the upper assembly 11. The upper ring 22 has an inner diameter corresponding to the outer diameter of the neck 85 of the upper cup 82, such that the upper ring 22 fits loosely around the neck 85. Below the upper ring 22 is the spacer 24, and below the spacer 24 is the ring 52 of the lower assembly 12. The ring 52 has an inner diameter corresponding to the outer diameter of the neck 85 of the upper cup 82, such that the ring 52 fits loosely around the neck 85. Below the ring 52 is the other spacer 25, and below that spacer 25 is the lower ring 23 of the upper assembly 11. The lower ring 23 has an inner diameter corresponding to the outer diameter of the neck 85 of the upper cup 82, such that the lower ring 23 fits loosely around the neck 85. Below the lower ring 23 is the lower cup 83, defining a lower bound of the annular hold 90 of the pivot housing 81.

The upper and lower rings 22 and 23, and the spacers 24 and 25, and the ring 25 are all mounted in the pivot housing 81. These elements are confined vertically by the upper and lower cups 82 and 83 and are prevented from lateral displacement by interaction with the neck 85 of the upper cup 82.

Within the pivot housing 81, the upper and lower rings 22 and 23 move independently of the ring 52 and of the spacers 24 and 25. As such, the upper and lower rings 22 and 23 rotate freely with respect to the ring 52, and so the upper and lower assemblies 11 and 12 coupled to the rings 22 and 23, and the ring 52, respectively, may pivot freely with respect to each other. This defines a first arrangement of the kit 10, which enables unrestricted pivotal movement of the upper and lower assemblies 11 and 12 with respect to each other, and about the central axis Y. Moreover, the worm screw 76 prevents movement of the ring 52, and thus disables adduction and abduction of the upper and lower assemblies 11 and 12 with respect to each other. Still further, because the upper assembly 11 includes the upper and lower rings 22 and 23 which are integral to the arm 21, the upper and lower rings 22 and 23 are disabled from movement with respect to the arm 21, and since the upper and lower rings 22 and 23 are carried within the pivot housing 81, the upper assembly 11 is disabled from adduction and abduction with respect to the pivot assembly 13.

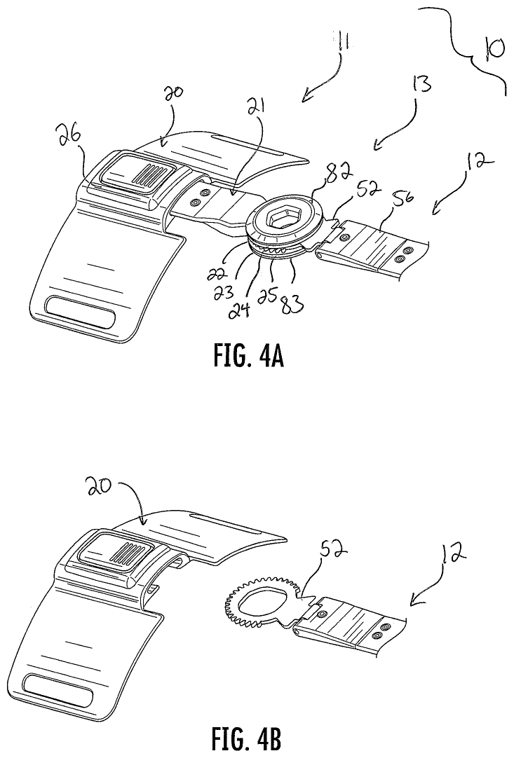

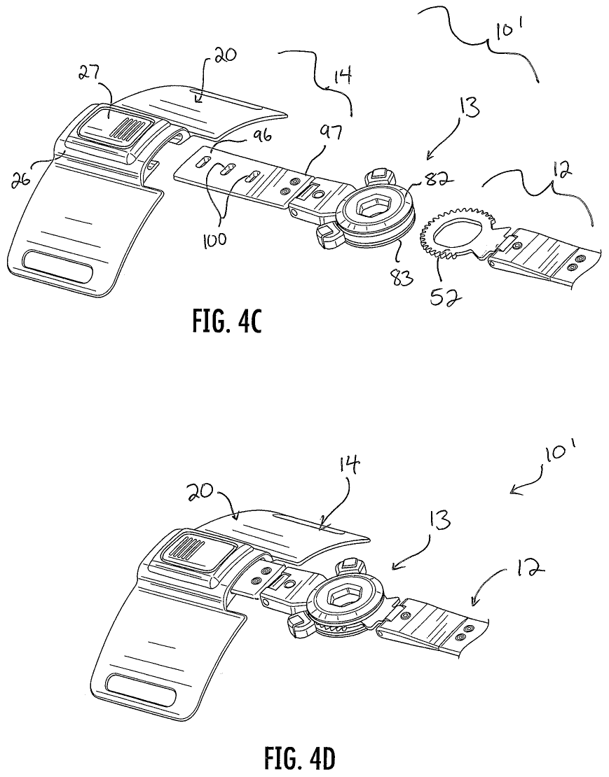

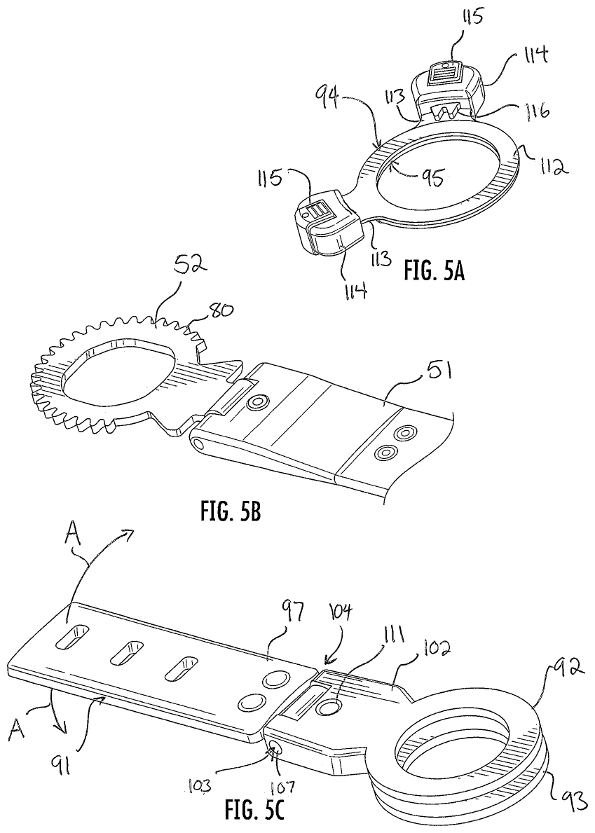

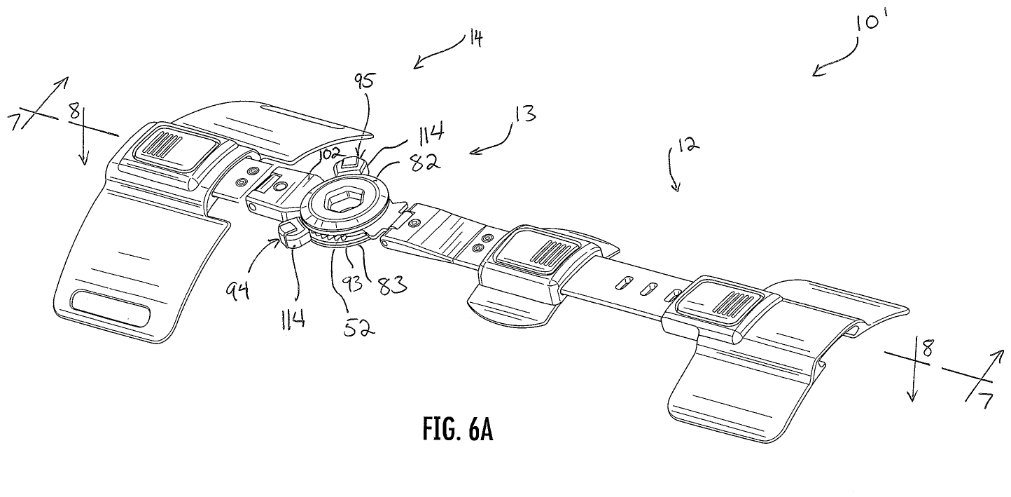

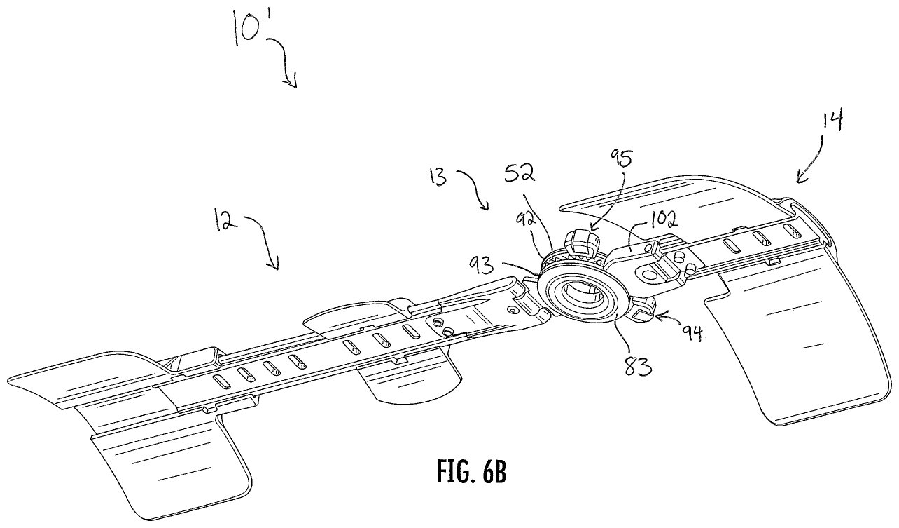

The teeth 80 on the ring 52 of the lower assembly 12 do not limit pivotal movement of the kit 10. However, the kit 10 can be modified to achieve that different functionality. FIGS. 1A-3B show the kit 10 in the first arrangement. FIGS. 6A-8 show the kit 10 in a second arrangement. FIGS. 4A-5C show various steps of replacing elements of the kit 10 to place it in the second arrangement. The second arrangement of the kit 10 provides functionality different from that of the first arrangement of the kit 10. As such, the reference character of the kit 10 is modified with a prime (“′”) symbol to denote the modified version of the kit 10.

FIG. 4A is an enlarged view of the upper assembly 11, the upper end 56 of the lower assembly 12, and the pivot assembly 13. To modify the kit 10, the upper assembly 11 is removed. The upper cup 82 of the pivot housing 81 is threadably disengaged from the lower cup 83, the upper cup 82 is removed, and the lower cup 83 is removed. This leaves the upper and lower rings and 23 and the spacers 24 and 25 of the upper assembly 11 disposed over, but no longer engaged to, the ring 52. The arm 21 is disengaged from the base 20 by unlocking the buckle 26, and the arm 21 is removed from the kit 10, thereby also removing the upper and lower rings 22 and 23 and the spacers 24 and 25.

This leaves the base 20 of the upper assembly 11 and the lower assembly 12, as shown in FIG. 4B. The pinion ring 52 remains at the end of the arm 56 of the lower assembly 12. Another upper assembly 14 can now be formed. The elements of the upper assembly 14 are shown in FIG. 4C. The upper assembly 14 includes the base 20, an arm 91 extending downward from the base 20 and terminating at upper and lower rings 92 and 93 opposite the base 20, to be carried within the pivot assembly 13. The upper assembly 14 further includes upper and lower set rings 94 and 95. The arm 91, like the arm 21, slides into and engages with the buckle 26 to be releasably secured in the base 20.

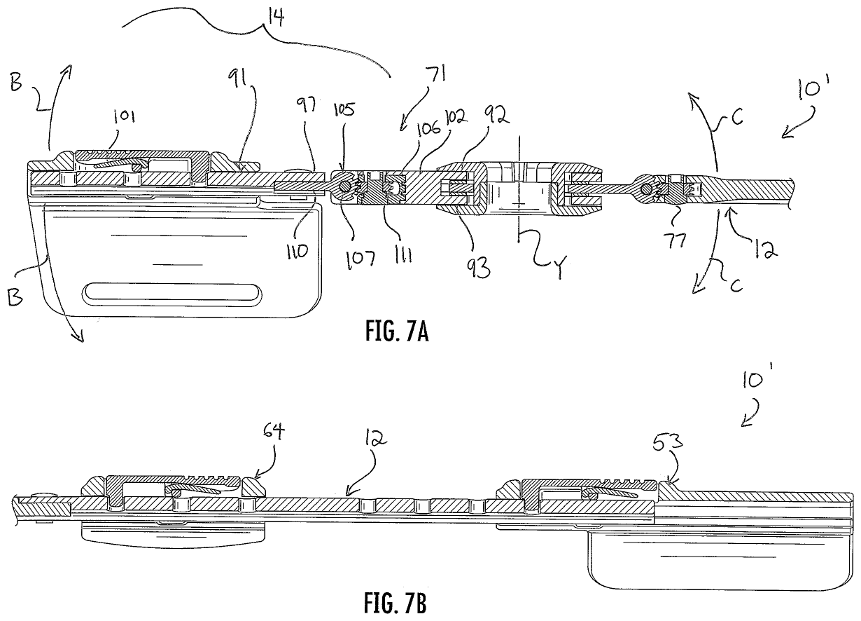

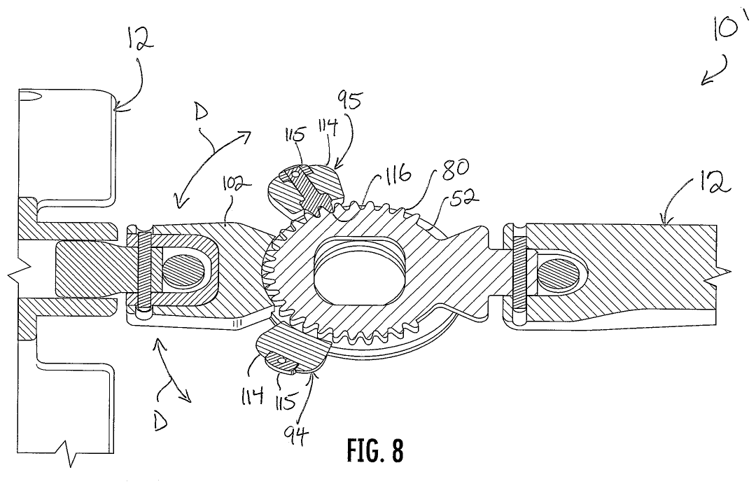

The arm 91 extends between the pivot assembly 13 and the base 20 to set the desired offset location of the pivot assembly 13 with respect to the base 20. The arm 91 is thin, elongate, and rigid. It includes an upper end 96 and an opposed lower end 97. Several spaced-apart slots 100 are formed through the upper end 96 of the arm 91. The tooth 34 on the underside of the lower end of the button 27 projects inwardly through one of the slots 100 to hold the arm 91 with respect to the buckle 26. The upper end of the button 27 can be depressed to pivot the button 27 and lift the tooth 34, which disengages the arm 91 from the buckle 26; the arm 91 can then be moved upward or downward, as desired, within the buckle 26 to change the effective length of the arm 91 and thereby move the pivot assembly 13 with respect to the base 20. A flat spring 101 (shown only in FIG. 7A) is compressed between the upper end 96 of the arm 91 and the button 27; the flat spring 101 biases the button 27 into the engaged position so that the button 27 does not inadvertently come loose and disengage the arm 91.

Referring now primarily to FIGS. 5C and 7A, the lower end 97 of the arm 91 terminates bluntly but is attached to the upper and lower rings 92 and 93. The upper and lower rings 92 and 93 are coupled to the lower end 97 via an articulating joint. The upper and lower rings 92 and 93 are coaxial, coextensive, parallel to each other, and vertically spaced apart from each other defining a gap therebetween. The rings 92 and 93 are formed integrally to and project from a base 102. Opposite the rings 92 and 93, the base 102 is formed with a cylindrical slot 103 which extends transverse to the length of the arm 91. An adjustment assembly 104 is carried at this slot 103.

The adjustment assembly 104 includes a knuckle 105 and a worm screw 106 engaged with each other. The knuckle 105 is mounted on a pin 107 carried in the slot 103. An arm 110 extends backward from the knuckle 105 and is secured to the underside of the lower end 97 of the arm 91, as shown in FIG. 7A. The knuckle 105 has teeth directed toward the worm screw 106, and the worm screw 106 has complemental threads which engage with the teeth on the knuckle 105.

The worm screw 106 is mounted on an adjustment knob 111, which extends out of a hole in the base 102 where it can be accessed. Rotating the knob 111 in one direction or another imparts rotational movement to the worm screw 106, whose threads engage with the teeth on the knuckle 105 and thereby pitch the knuckle 105 up or down. Because the arm 91 is rigidly connected to the knuckle 105, pitching movement of the knuckle 105 imparts pitching movement to the arm 91. The rings 92 and 93 are applied to the pivot assembly 13, and so pitching movement of the rings 92 and 93 causes the upper assembly 14 to move into and out of the plane of the pivot assembly 13, as indicated by the arrowed lines A in FIG. 5C. Thus, the adjustment assembly 104 can be adjusted to set a desired angle of the upper assembly 14 with respect to the pivot assembly 13, thereby enabling and defining a desired degree of hip abduction or adduction.

The upper and lower set rings 94 and 95 are shown in FIG. 5A. The set rings 94 and 95 are identical, and so the description herein will refer to either one without distinction, and as is convenient for the structure shown in FIG. 5A, but with the understanding that the description applies equally to both. The set ring 94 has an annular body 112 and a prong 113 extending radially outward from the body 112. The set ring 94 has an inner diameter which corresponds to the inner diameters of the spacers 24 and 25 and the ring 52. A cap 114 is fit over the prong 113; the cap 114 holds a button 115 which is coupled to a tooth 116. The tooth 116 is parallel to and offset from the plane of the body 112, but is directed radially inward, toward the body 112 and the hole bound by the body 112. The tooth 116 is moveable between the advanced position shown in FIG. 5A, and a retracted position pulled away from the body 112. A spring is carried within the cap 114 biasing the tooth 116 toward the advanced position. To move the tooth 116 from the advanced position to the retracted position, the button 115 is depressed and pulled back, away from the body 112 of the ring 94. This draws the tooth 116 away from the body 112, which is useful for limiting the range of pivotal movement, as is described later. The tooth 116 on the lower set ring 95 is above the prong 113; the tooth 116 on the upper set ring 94 is below the prong 113. The teeth 116 on the set rings 94 and 95 engage with the teeth 80 on the ring 52 of the lower assembly 12.

The set rings 94 and 95 are carried in the pivot housing 81 with the upper and lower rings 92 and 93 of the upper assembly 14. The upper and lower rings 92 and 93 are registered above and below, respectively, the set rings 94 and 95. The set rings 94 and 95 define a small and thin holding space between the upper and lower rings 96 and 97. The ring 52 of the lower assembly occupies this holding space; the set rings 94 and 95 are registered above and below, respectively, the ring 52. In this way, the teeth 116 of the set rings 94 and 95 are disposed to engage with the teeth 80 on the ring 52 to selectively adjust and index rotation of the upper and lower assemblies 14 and 12 with respect to each other.

FIG. 4C shows a partially exploded view of the kit 10′ with the upper and lower assemblies 14 and 12. Generally, the upper cup 82 would not yet be applied to the lower cup 83, so that the ring 52 could be slipped between the set rings 94 and 95. FIG. 4D shows the kit 10′ assembled with the upper and lower assemblies 14 and 12 mounted for movement with respect to each other at the pivot assembly 13; the ring 52 is now properly received between the set rings 94 and 95.

FIGS. 6A-8 show the modified kit 10′ in greater detail. The upper and lower assemblies 14 and 12 are brought together so that the ring 52 of the lower assembly 12 is registered with and between the upper and lower rings 92 and 93 and the set rings 94 and 95. The caps 114 are proximate the base 102 of the upper assembly 14, and, although not visible in FIGS. 6A-8, the teeth 116 of the set rings 94 and 95 are engaged with the ring 52. The lower cup 83 is placed under the lower ring 93, with its threaded neck 87 extending upward through the interior hollow formed by the upper and lower rings 92 and 93. The upper cup 82 is threadably engaged onto the lower cup 83, thereby forming the pivot housing 81.

FIGS. 7A and 7B show section views bisecting the kit 10′. The upper assembly 14 may be adjusted and set with defined abduction and adduction amounts in the direction of the arrowed lines B. Similarly, the lower assembly 12 may be adjusted and set with defined abduction and adduction amounts in the direction of the arrowed lines C. In this second arrangement of the kit 10′, adduction and abduction of the upper and lower assemblies 14 and 12 is enabled. Moreover, the upper assembly 14 is independently adjustable and enabled for adduction and abduction with respect to the pivot assembly 13, and the lower assembly 12 is also independently adjustable and enabled for adduction and abduction with respect to the pivot assembly 13. Thus, an attending physician or health worker, or the patient themself, may precisely set the amount and location of abduction or adduction for the patient by adjusting the knobs 77 and 111.

FIG. 8 is another section view, showing the engagement of the teeth 116 of the set rings 94 and 95 with the teeth 80 of the ring 52. The set rings 94 and 95 can be positioned to limit the amount of pivotal movement, or flexion and extension, in the directions indicated by arrowed lines D. In this illustration, the set rings 94 and 95 allow a limited range of movement, provided by the angular spacing of the caps 114 from the base 102. However, the attending physician or health worker, or the patient themself, may depress the buttons 115 on the caps 114, move the teeth 116 into the retracted positions, and adjust the set rings 94 and 95 around the pinion ring 52. By moving the either or both of the set rings 94 and 95 around the ring 52, the kit 10′ changes the extent of unrestricted movement.

Moving the caps 114 further away from the base 102 allows the patient to flex or extend his hip with a larger range of movement. The set rings 94 and 95 limit that range, however; they stop such flexion or extension movement when the lower assembly 12 pivots with respect to the upper assembly 14 to the extent that at least one of the caps 114, locked with respect to the pinion ring 52, rotates around and comes into contact with the base 102 of the upper assembly 14. The base 102 prevents further rotational movement of the cap 114 and thus prevents further pivoting of the lower assembly, thereby limiting flexion and extension.

A preferred embodiment is fully and clearly described above so as to enable one having skill in the art to understand, make, and use the same. Those skilled in the art will recognize that modifications may be made to the description above without departing from the spirit of the specification, and that some embodiments include only those elements and features described, or a subset thereof. To the extent that modifications do not depart from the spirit of the specification, they are intended to be included within the scope thereof.

What is claimed is:

1. A hip brace kit comprising:
a first assembly for retention against a limb, the first assembly comprising a first base and a first arm terminating opposite the first base at concentric first rings;
a second assembly for retention against the limb, the second assembly comprising a second base and a second arm terminating opposite the second base at a second ring;
a third assembly for retention against the limb, the third assembly comprising a third base and a third arm terminating opposite the third base at a third ring;
a pivot assembly including an upper cup and a lower cup engageable to each other to define a pivot housing with an annular hold, the hold defining a central axis;
a first arrangement of the hip brace kit, in which the first assembly and the second assembly are mounted in the pivot assembly to enable unrestricted pivotal movement of the first and second assemblies with respect to each other about the central axis and to disable adduction and abduction of the first and second assemblies with respect to each other; and
a second arrangement of the hip brace kit, in which the first assembly and the third assembly are mounted in the pivot assembly to disable unrestricted pivotal movement of the first and third assemblies with respect to each other about the central axis and to enable adduction and abduction of the first and third assemblies with respect to each other.

2. The hip brace kit of claim 1, wherein the second ring is a pinion having a plurality of outwardly-directed teeth formed on a circumference thereof.

3. The hip brace kit of claim 2, wherein the third assembly further comprises set rings, each having inwardly-directed teeth which, when the set rings are placed within the hold, engage with the outwardly-directed teeth on the second ring.

4. The hip brace kit of claim 3, wherein the rings are moveable about the pinion to adjustably index the inwardly-directed teeth of the set rings with the outwardly-directed teeth of the pinion and thereby disable the unrestricted pivotal movement.

5. The hip brace kit of claim 1, wherein the third ring is connected to the third arm at an adjustment assembly including:
a knuckle carrying outwardly-directed teeth; and
a worm screw on the third ring, the worm screw engaged with the teeth of the knuckle, wherein the worm screw is mounted for rotational movement imparting transverse adduction or abduction of the third arm with respect to the third ring.

6. The hip brace kit of claim 1, wherein:
the first rings are vertically spaced apart from each other; and
the third rings are vertically spaced apart from each other.

7. The hip brace kit of claim 1, further comprising spacers disposed between the first rings and the second ring.

8. A hip brace kit comprising:
a first assembly for retention against a limb, the first assembly comprising a first base and a first arm terminating opposite the first base at a first pivot connection defined by concentric first rings;
a second assembly for retention against the limb, the second assembly comprising a second base and a second arm terminating opposite the second base at a second pivot connection defined by a second ring;
a third assembly for retention against the limb, the third assembly comprising a third base and a third arm terminating opposite the third base at a third pivot connection defined by a third ring;
a pivot assembly including an upper cup and a lower cup engageable to each other to define a pivot housing with an annular hold, the hold defining a central axis;
a first arrangement of the hip brace kit, in which the first assembly and the second assembly are mounted in the pivot assembly to enable unrestricted pivotal movement of the first and second assemblies with respect to each other about the central axis; and
a second arrangement of the hip brace kit, in which the first assembly and the third assembly are mounted in the pivot assembly to disable unrestricted pivotal movement of the first and third assemblies with respect to each other about the central axis.

9. The hip brace kit of claim 8, wherein the second ring is a pinion having a plurality of outwardly-directed teeth formed on a circumference thereof.

10. The hip brace kit of claim 9, wherein the third assembly further comprises set rings, each having inwardly-directed teeth which, when the set rings are placed within the hold, engage with the outwardly-directed teeth on the second ring.

11. The hip brace kit of claim 10, wherein the rings are moveable about the pinion to adjustably index the inwardly-directed teeth of the set rings with the outwardly-directed teeth of the pinion and thereby disable the unrestricted pivotal movement.

12. The hip brace kit of claim 8, wherein the third ring is connected to the third arm at an adjustment assembly including:
a knuckle carrying outwardly-directed teeth; and
a worm screw on the third ring, the worm screw engaged with the teeth of the knuckle, wherein the worm screw is mounted for rotational movement imparting transverse adduction or abduction of the third arm with respect to the third ring.

13. The hip brace kit of claim 8, wherein:
the first rings are vertically spaced apart from each other; and
the third rings are vertically spaced apart from each other.

14. The hip brace kit of claim 8, further comprising spacers disposed between the first rings and the second ring.

15. A hip brace kit comprising:
a first assembly for retention against a limb, the first assembly comprising a first base and a first arm terminating opposite the first base at a first pivot connection defined by concentric first rings;
a second assembly for retention against the limb, the second assembly comprising a second base and a second arm terminating opposite the second base at a second pivot connection defined by a second ring;
a third assembly for retention against the limb, the third assembly comprising a third base and a third arm terminating opposite the third base at a third pivot connection defined by a third ring;
a pivot assembly including an upper cup and a lower cup engageable to each other to define a pivot housing with an annular hold, the hold defining a central axis;
a first arrangement of the hip brace kit, in which the first assembly and the second assembly are mounted in the pivot assembly to disable adduction and abduction of the first and second assemblies with respect to each other; and
a second arrangement of the hip brace kit, in which the first assembly and the third assembly are mounted in the pivot assembly to enable adduction and abduction of the first and third assemblies with respect to each other.

16. The hip brace kit of claim 15, wherein the second ring is a pinion having a plurality of outwardly-directed teeth formed on a circumference thereof.

17. The hip brace kit of claim 16, wherein the third assembly further comprises set rings, each having inwardly-directed teeth which, when the set rings are placed within the hold, engage with the outwardly-directed teeth on the second ring.

18. The hip brace kit of claim 17, wherein the rings are moveable about the pinion to adjustably index the inwardly-directed teeth of the set rings with the outwardly-directed teeth of the pinion and thereby disable the unrestricted pivotal movement.

19. The hip brace kit of claim 15, wherein the third ring is connected to the third arm at an adjustment assembly including:
a knuckle carrying outwardly-directed teeth; and
a worm screw on the third ring, the worm screw engaged with the teeth of the knuckle, wherein the worm screw is mounted for rotational movement imparting transverse adduction or abduction of the third arm with respect to the third ring.

20. The hip brace kit of claim 15, wherein:
the first rings are vertically spaced apart from each other; and
the third rings are vertically spaced apart from each other.
